(19) United States
(12) Patent Application Publication
(10) Pub. No.: US 2021/0119740 A1
(43) Pub. Date: Apr. 22, 2021

(54) SYSTEM AND METHOD FOR SIGNAL SENSING

(71) Applicant: Industrial Technology Research Institute, Hsinchu (TW)

(72) Inventors: Ting-Wu Ho, Hsinchu City (TW); Chia-Lung Liu, Hsinchu County (TW); Fu-Te Hsu, Taoyuan City (TW)

(73) Assignee: Industrial Technology Research Institute, Hsinchu (TW)

(21) Appl. No.: 17/136,004

(22) Filed: Dec. 29, 2020

Related U.S. Application Data
(63) Continuation-in-part of application No. 16/726,948, filed on Dec. 26, 2019.
(60) Provisional application No. 62/876,788, filed on Jul. 22, 2019.

(30) Foreign Application Priority Data
Nov. 8, 2019 (TW) 108140738

(51) Int. Cl. H04L 5/00; G01S 3/14; H04B 17/309; G01S 3/74; H04L 27/26

(52) U.S. Cl. CPC H04L 5/0007; G01S 3/14; H04L 27/2602; H04B 17/309; G01S 3/74; H04L 5/0035

(57) ABSTRACT

A system and a method for signal sensing are provided. The system for signal sensing includes a sensing device, a processor and a time server coupled to the sensing device. The sensing device includes a plurality of receivers and an oscillator coupled to the receivers. The receivers are synchronized by receiving a time synchronization signal from the time server. The receivers monitor a plurality of signals of an object according to a clock generated by the oscillator and obtains a plurality of channel state information (CSI) according to the signals. The processor calculates an angle of arrival of the signals according to the CSI.

### CROSS-REFERENCE TO RELATED APPLICATION

This application is a continuation-in-part application of and claims the priority benefit of U.S. application Ser. No. 16/726,948, filed on Dec. 26, 2019, now pending. The prior U.S. application Ser. No. 16/726,948 claims the priority benefit of U.S. provisional application Ser. No. 62/876,788, filed on Jul. 22, 2019, and Taiwan application serial no. 108140738, filed on Nov. 8, 2019. The entirety of each of the above-mentioned patent applications is hereby incorporated by reference herein and made a part of this specification.

### BACKGROUND

### Technical Field

The disclosure relates to a system and a method for signal sensing, and more particularly to a system and a method for signal sensing based on orthogonal frequency-division multiplexing (OFDM) technology.

### Description of Related Art

Radar sensing technology is widely used in a variety of different sensing fields, for example, health care, safety monitoring, smart home, food safety enforcement, and other applications. Existing radar sensing devices (for example, doppler radar, millimeter wave (mmWave) radar, etc.) are too costly. Considering that a consumer will hesitate in making a purchase due to price considerations, using a cheap OFDM device (for example, a device using technologies such as WiFi, LTE, 5G, etc.) for sensing has been one of the most popular research techniques in recent years.

In an application of indoor positioning, OFDM devices may employ a sniffer mechanism to perform indoor positioning. The signal of an target object monitored by the sniffer mechanism must include channel state information (CSI) of multiple antennas to calculate the angle of arrival (AoA) of the signal to perform indoor positioning. However, methods mentioned above still have two key issues to overcome.

[Issue 1: OFDM Device Providing CSI Only Support a Single Antenna]

An OFDM device only support CSI information collection through a single antenna. When multiple OFDM devices are used to receive a signal, a problem of frequency offset may occur on the receiving ends because each receiving end belong to different OFDM devices and positioning error may possibly occur.

[Issue 2: There is No Time Synchronization Mechanism Between the OFDM Devices]

When multiple OFDM devices are used to receive a signal of a target object, the time point of a packet transmitted by the target object arriving the OFDM devices are different. CSI of the same packet must be used to calculate the AoA of the signal.

### SUMMARY

The disclosure provides a system and a method for signal sensing, which solves the problem of frequency offset in the receiving ends and supports time synchronization mechanism between the OFDM devices, thereby improving the result of signal AoA calculation.

The disclosure provides a system for signal sensing. The system for signal sensing includes a sensing device, a processor and a time server coupled to the sensing device. The sensing device includes a plurality of receivers and an oscillator coupled to the receivers. The receivers are synchronized by receiving a time synchronization signal from the time server. The receivers monitor a plurality of signals of an object according to a clock generated by the oscillator and obtains a plurality of channel state information (CSI) according to the signals. The processor calculates an angle of arrival of the signals according to the CSI.

The disclosure provides a method for signal sensing used in the system for signal sensing. The system for signal sensing includes a sensing device, a processor and a time server. The sensing device includes a plurality of receivers and an oscillator coupled to the receivers. The method for signal sensing includes: synchronizing the receivers by receiving a time synchronization signal from the time server; monitoring, by each of the receivers, a plurality of signals of an object according to a clock generated by the oscillator and obtains a channel state information (CSI) according to the signals; and calculating an angle of arrival of the signals according to the CSI by the processor.

Based on the above, the system and the method for signal sensing of the disclosure integrates multiple receivers based on OFDM technology into the same sensing device and allow the receivers to share the same oscillator, thereby solving frequency offset problem between the receivers. In addition, the disclosure also synchronizes the receivers by receiving a time synchronization signal from the time server such that CSI of the same packet can be used to calculate the AoA of the signal, thereby improving the accuracy of the AoA estimation result.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram of a system for signal sensing according to an embodiment of the disclosure.

FIG. 2 is a schematic diagram of a module for signal sensing according to an embodiment of the disclosure.

FIG. 3 is a schematic diagram of a system for signal sensing according to an embodiment of the disclosure.

FIG. 4 is a schematic diagram of a sensing device according to an embodiment of the disclosure.

FIG. 5 is a schematic diagram of an example of frequency offset problem according to an embodiment of the disclosure.

FIG. 6A to FIG. 6F are schematic diagrams of an AoA estimation example according to an embodiment of the disclosure.

FIG. 7 is a schematic diagram of a method for signal sensing according to an embodiment of the disclosure.

### DESCRIPTION OF THE EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

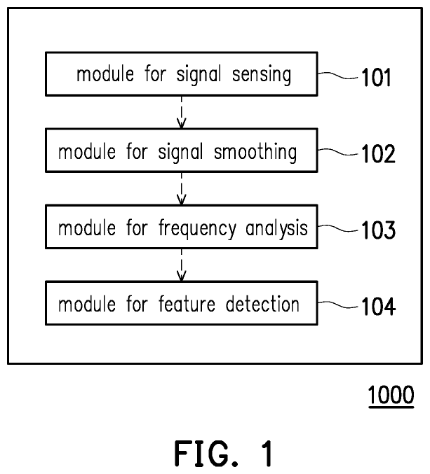

FIG. 1 is a schematic diagram of a system for signal sensing according to an embodiment of the disclosure.

Referring to FIG. 1, a system for signal sensing 1000 mainly includes a module for signal sensing 101, a module for signal smoothing 102, a module for frequency analysis 103, and a module for feature detection 104.

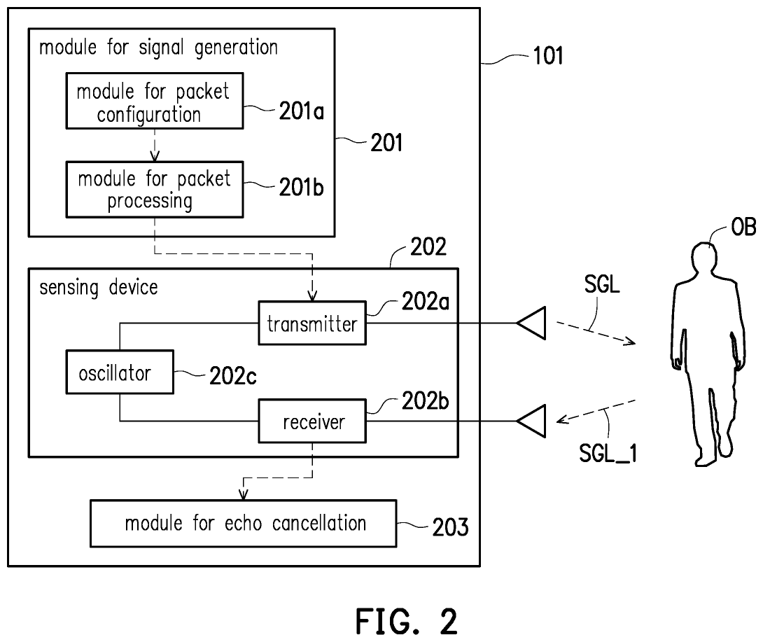

FIG. 2 is a schematic diagram of a module for signal sensing according to an embodiment of the disclosure.

Referring to FIG. 2, the module for signal sensing 101 in FIG. 1 includes a module for signal generation 201, a sensing device 202, and a module for echo cancellation 203. The module for signal generation 201 includes a module for packet configuration 201a and a module for packet processing 201b. The sensing device 202 includes a transmitter 202a, a receiver 202b, and an oscillator 202c.

In the embodiment, the system for signal sensing 1000 further includes a processor (not shown) and a storage circuit (not shown). The processor is coupled to the storage circuit and the sensing device 202. A plurality of code segments are stored in the storage circuit of the signal sensing circuit 1000. After the code segments are installed, the code segments are executed by the processor. For example, a plurality of modules are included in the storage circuit. Various operations of the module for packet configuration 201a, the module for packet processing 201b, the module for echo cancellation 203, the module for signal smoothing 102, the module for frequency analysis 103, and the module for feature detection 104 are respectively executed by the modules, wherein each module is formed by one or more code segments. However, the disclosure is not limited thereto. The various operations of the module for packet configuration 201a, the module for packet processing 201b, the module for echo cancellation 203, the module for signal smoothing 102, the module for frequency analysis 103, and the module for feature detection 104 may also be implemented by using other hardware forms.

In particular, the transmitter 202a and the receiver 202b of the disclosure may be a transceiver (or a circuit) based on orthogonal frequency-division multiplexing (OFDM) technology.

The oscillator 202c is coupled to the transmitter 202a and the receiver 202b. The oscillator 202c is configured to generate a clock signal compliant with the specifications and is simultaneously provided to the transmitter 202a and the receiver 202b as an oscillation source. In the embodiment, the transmitter 202a and the receiver 202b share the clock signal generated by the oscillator 202c.

In the embodiment, the module for signal generation 201 is configured to transmit a plurality of packets according to a packet configuration information to generate a signal. In more details, the module for packet configuration 201a in the module for signal generation 201 receives the packet configuration information set by a user or a device. The packet configuration information may be the transmission frequency of the packet. The module for packet processing 201b may, for example, cut the data to be sent into a plurality of packets according to the packet configuration information and transmit the plurality of packets to generate a signal to be transmitted through the transmitter 202a.

Then, the transmitter 202a generates a plurality of subcarriers orthogonal to each other based on OFDM operation principle, divides the signal from the module for packet processing 201b into a plurality of subsignals, and respectively modulates the plurality of subsignals according to the plurality of subcarriers to generate a plurality of output signals. Next, the transmitter 202a transmits an output signal SGL according to the packet configuration information and the clock signal of the oscillator 202c.

Thereafter, the receiver 202b receives at least one output signal SGL_1 (also referred to as a first output signal) reflected via an object OB in the output signal SGL according to the clock signal of the oscillator 202c. For example, the receiver 202b receives the output signal SGL_1 in the analog signal form according to the clock signal of the oscillator 202c and samples the output signal SGL_1 in the digital signal form.

After obtaining the output signal SGL_1, the receiver 202b obtains a channel state information according to the output signal SGL_1. The processor of the system for signal sensing 1000 identifies a state of the object OB according to the channel state information and outputs the state of the object.

In more details, in the operation of obtaining the channel state information according to the output signal SGL_1, the interference signal in the output signal SGL_1 may be first cancelled through the module for echo cancellation 203. In particular, the interference signal is transmitted via a path (also referred to as a first path) between the transmitter 202a and the receiver 202b, and the first path is not reflected via the object OB. In other words, based on the multipath issue of wireless transmission, parts of the signals transmitted by the transmitter 202a are directly transmitted from the transmitter 202a to the receiver 202b without being reflected and these signals cause error in terms of judgment. Therefore, these signals are identified as interference signals. The method of the module for echo cancellation 203 for cancelling the interference signal may be a hardware method, the multiple reference active noise control (multiple reference ANC), the recursive least squares (RLS), the least mean square (LMS), the filtered-x LMS, (FxLMS), etc.

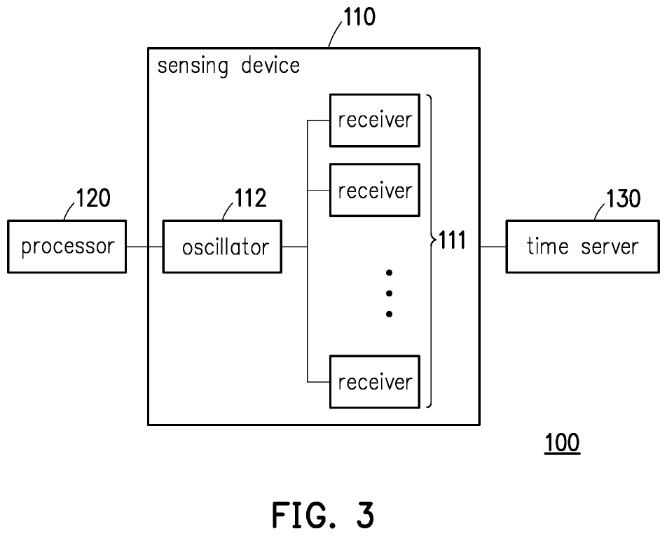

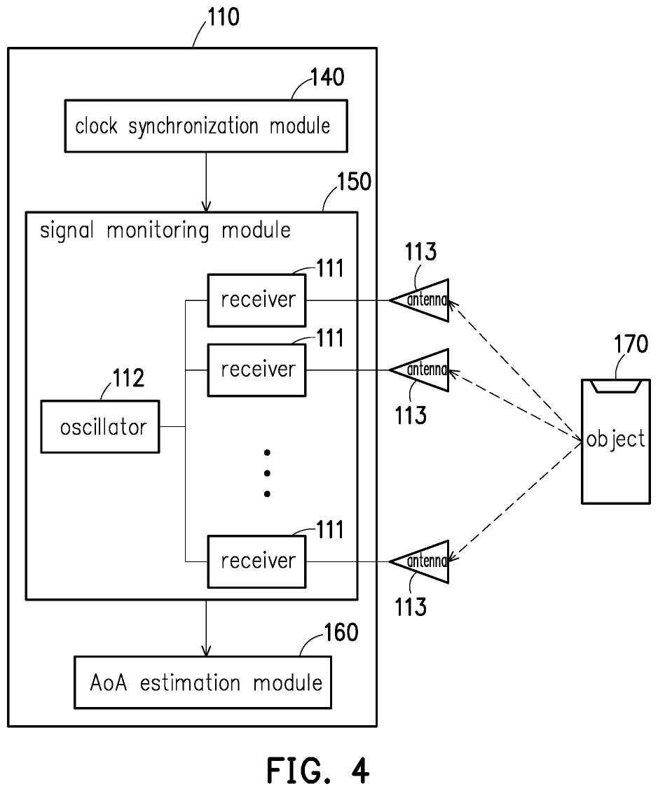

FIG. 3 is a schematic diagram of a system for signal sensing according to an embodiment of the disclosure. FIG. 4 is a schematic diagram of a sensing device according to an embodiment of the disclosure.

Referring to FIG. 3 and FIG. 4, the system for signal sensing 100 includes a sensing device 110, a processor 120 coupled to the sensing device 110 and a time server 130 coupled to the sensing device 110. In an embodiment, the processor 120 may be included in the sensing device 110. The sensing device 110 includes multiple receivers 111 and an oscillator 112 coupled to the receivers 111. The oscillator 112 is configured to generate a clock signal compliant with the specifications and is simultaneously provided to the multiple receivers 111 as an oscillation source. In the embodiment, the multiple receivers 111 share the clock signal generated by the oscillator 112. The receivers 111, for example, is an Orthogonal Frequency Division Multiplexing (OFDM) receiver based on OFDM technology and includes a microcontroller (not shown). The microcontroller, for example, includes a 160 or 240 MHz microprocessor and a 520 KB SRAM. An antenna 113 may be integrated with each of the receivers 111.

In an embodiment, the sensing device 110 further includes a storage circuit (not shown) coupled to the processor 120. A plurality of code segments may be stored in the storage circuit of the sensing device 110. The code segments corresponding to the clock synchronization module 140, the signal monitoring module 150 and the AoA estimation module 160 may be executed by the processor 120 and/or the microcontroller of the receivers 111. However, the present disclosure is not limited thereto. The clock synchronization module 140, the signal monitoring module 150 and the AoA estimation module 160 may also be implemented by using other hardware forms or the combination of hardware and software/firmware forms.

In an embodiment, when the receivers 111 are booted up, the receivers 111 may be configured to a client mode by the clock synchronization module 140 and the receivers 111 may automatically connect to the time server 130 to perform a time synchronization procedure. The receivers 111 may receive a time synchronization signal from the time server 130 by a network time protocol (NTP), such that all the receivers 111 are synchronized. After all the receivers 111 are synchronized, the receivers 111 may be configured to a sniffer mode from the client mode to monitor the signals in an environment including a plurality of channel state information (CSI) packets having absolute time information.

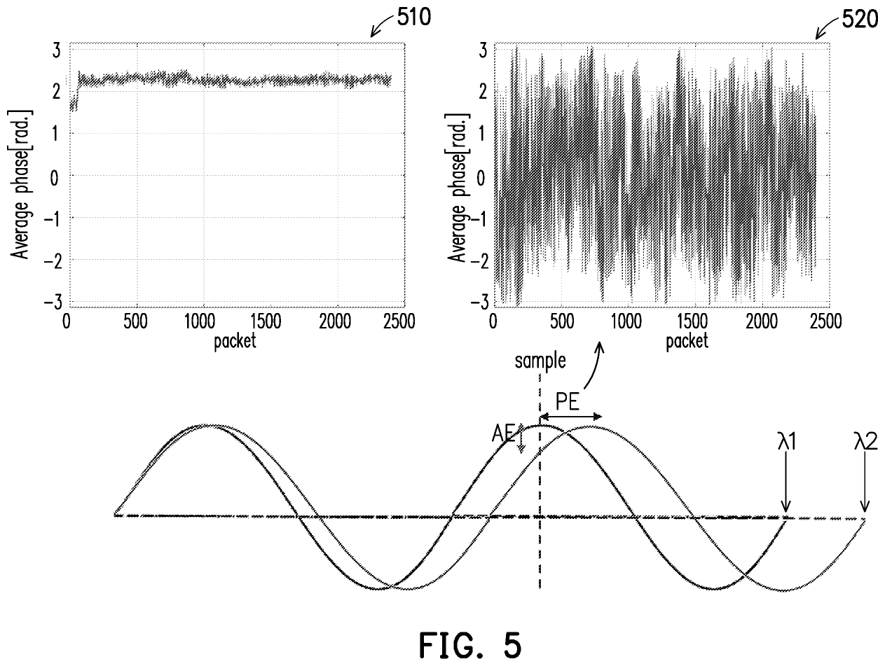

In the signal monitoring module 150, the receivers 111 in the sensing device 110 couple to the same oscillator 112 to receive the signal from an object 170 through the antennas 113, such that the frequency offset problem can be overcome. For example, in the LTE-A technology, the frequency offset may be ±50 ppb in wide area, ±100 ppb in local area and ±250 ppb at home. In the 802.11 technology, the frequency offset may be ±25 ppm under 2.4 GHz (i.e., 2.4 GHz±60 KHz) and ±20 under 5.8 GHz (i.e., 5.8 GHz±116 KHz). The wavelength of a signal may change by the effect of the frequency offset and the phase error and the amplitude error may occur, which may result in error of positioning. FIG. 5 is a schematic diagram of an example of frequency offset problem according to an embodiment of the disclosure. Referring to FIG. 5, chart 510 illustrates an average phase of 64 subcarriers sharing the oscillator 112 according to an embodiment of the present disclosure and chart 520 illustrates an average phase of 64 subcarriers without sharing any oscillator. In the example, 40 packets are ping per second and 2400 packets are measured in total. In the case of chart 520, λ2 changed by the effect of frequency offset may be different from original 21 and the phase error PE and the amplitude error AE may occur due to frequency offset problem.

Referring back to FIG. 4, the signal monitoring module 150 may instruct the receivers 111 to monitor the signals of an object 170 according to a clock generated by the oscillator 112 and obtains a plurality of CSIs according to the signals. The CSIs are transmitted to the AoA estimation module 160 to calculate the AoA (Angle of Arrival) of the signals of the object 170. The AoA estimation module 160 may calculate the AoA of the signals according to the CSI by a MUSIC (i.e., Multiple Signal Classification) algorithm, a phase difference algorithm or other AoA calculation algorithm. For example, in the MUSIC algorithm, the AoA estimation module 160 receives the signals and calculate the correlation matrix Rₓₓ=E[XX^{H}]. Then, eigen decomposition Rₓₓvᵢ=λᵢvᵢ, i=1˜M is performed and subspace is defined by λᵢ, wherein λ₁≥λ₂≥ . . . ≥λ_{N+1}= . . . =λ_{M}, E_{S}=span{v₁, v₂, . . . , v_{N}} and E_{N}=span{v_{N+1}, v_{N+2}, . . . , v_{M}}. At last, the AoA estimation module 160 calculates MUSIC spectrum P_{MUSIC}(θ) and find N peaks of MUSIC spectrum, wherein P_{MUSIC}(θ)=1/(a^{H}(θ)E_{N}E_{N}^{H}a(θ)).

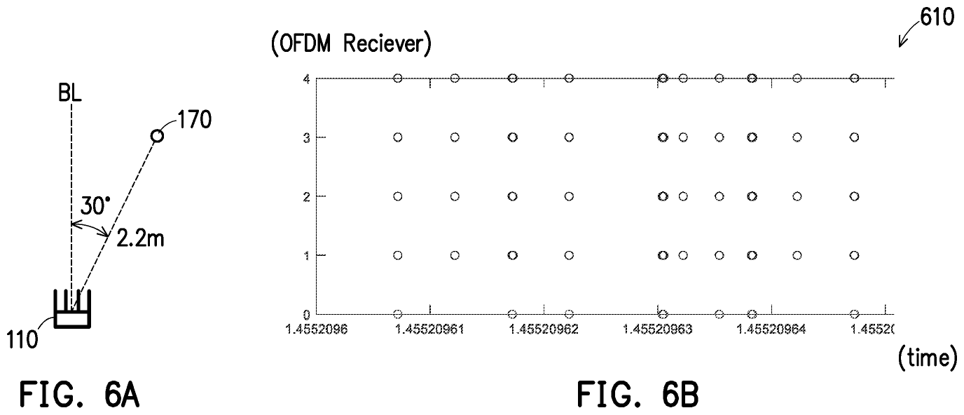

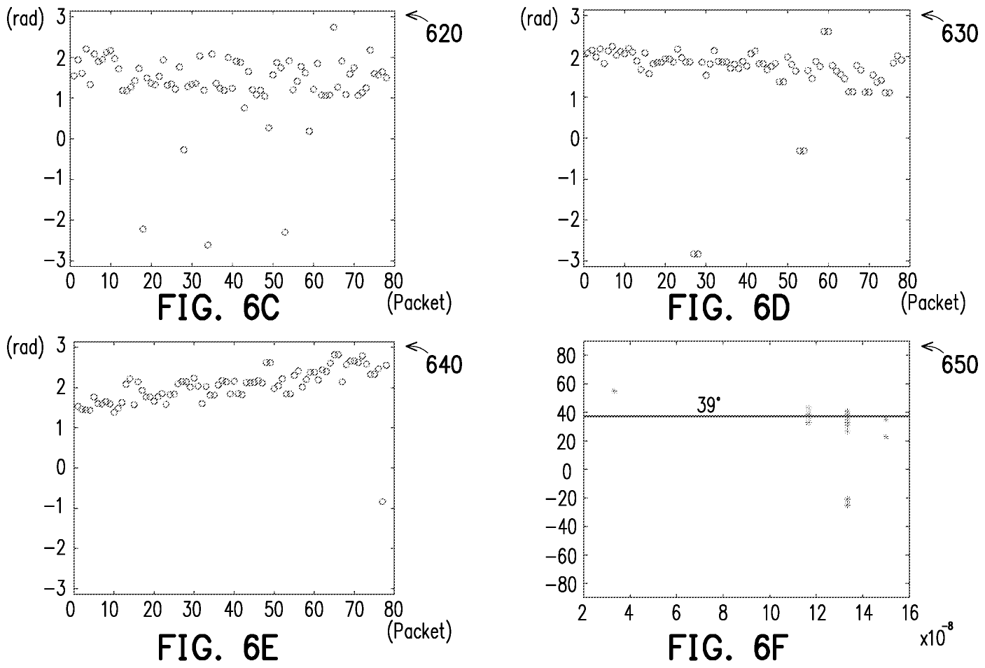

FIG. 6A to FIG. 6F are schematic diagrams of an AoA estimation example according to an embodiment of the disclosure.

Referring to FIG. 6A to FIG. 6F, the sensing device 110 monitors the signals transmitted by the object 170. The object 170, for example, is a cell phone and sends a ping AP signal. The distance between the sensing device 110 and the object 170 is 2.2 meters and the actual angel between the baseline BL of the sensing device 110 and the direction of the sensing device 110 transmitting the signal to the object 170 is 30 degrees. In this example, the sensing device 110 has 4 OFDM receivers, which are also called sniffer 1 to sniffer 4. Given that the OFDM receivers are synchronized by an external time server after booted up and the signals transmitted by the object 170 carries CSI packets having absolute time information, only when the 4 packets respectfully received by the 4 OFDM receivers are received within a predetermined time interval (for example, 10 ms) that the 4 packets are considered the same packet and used for AoA estimation, as shown in chart 610 of FIG. 6B. Chart 620 in FIG. 6C illustrates the phase difference between the packets received by sniffer 1 and sniffer 2. Chart 630 in FIG. 6D illustrates the phase difference between the packets received by sniffer 2 and sniffer 3. Chart 640 in FIG. 6E illustrates the phase difference between the packets received by sniffer 3 and sniffer 4. It can be shown in FIG. 6C to FIG. 6E that the phase difference between the packets received by adjacent sniffers are rather small. Finally, chart 650 in FIG. 6F shows that the AoA estimation result is 39 degrees.

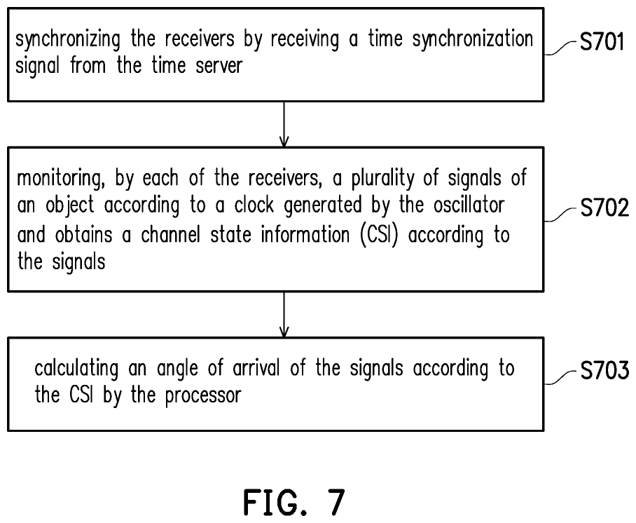

FIG. 7 is a schematic diagram of a method for signal sensing according to an embodiment of the disclosure.

Referring to FIG. 7, in step S701, synchronizing the receivers by receiving a time synchronization signal from the time server.

In step S702, monitoring, by each of the receivers, a plurality of signals of an object according to a clock generated by the oscillator and obtains a channel state information (CSI) according to the signals.

In step S703, calculating an angle of arrival of the signals according to the CSI by the processor.

Based on the above, the system and the method for signal sensing of the disclosure integrates multiple receivers based on OFDM technology into the same sensing device and allow the receivers to share the same oscillator, thereby solving frequency offset problem between the receivers. In addition, the disclosure also synchronizes the receivers by receiving a time synchronization signal from the time server such that CSI of the same packet can be used to calculate the AoA of the signal, thereby improving the accuracy of AoA estimation result.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A system for signal sensing, comprising:
a sensing device, comprising:
a plurality of receivers;
an oscillator, coupled to the receivers;
a processor, coupled to the sensing device; and
a time server, coupled to the sensing device, wherein
the receivers are synchronized by receiving a time synchronization signal from the time server,
the receivers monitor a plurality of signals of an object according to a clock generated by the oscillator and obtains a plurality of channel state information (CSI) according to the signals, and
the processor calculates an angle of arrival of the signals according to the CSI.

2. The system for signal sensing according to claim 1, wherein the receivers are configured to a client mode when booted up and receive the time synchronization signal from the time server by a network time protocol.

3. The system for signal sensing according to claim 2, wherein the receivers are configured to a sniffer mode from the client mode to monitor the signals, wherein the signals comprises a plurality of CSI packets having time information.

4. The system for signal sensing according to claim 3, wherein the processor calculates the angle of arrival according to the CSI packets received by the receivers in a predetermined time interval.

5. The system for signal sensing according to claim 1, wherein the processor calculates the angle of arrival by an algorithm, the algorithm comprising a MUSIC algorithm and a phase difference algorithm.

6. The system for signal sensing according to claim 1, wherein each of the receivers is an Orthogonal Frequency Division Multiplexing (OFDM) receiver.

7. A method for signal sensing used in a system for signal sensing, the system for signal sensing comprising a sensing device, a processor and a time server, the sensing device comprising a plurality of receivers and an oscillator coupled to the receivers, the method for signal sensing comprising:
synchronizing the receivers by receiving a time synchronization signal from the time server;
monitoring, by each of the receivers, a plurality of signals of an object according to a clock generated by the oscillator and obtains a channel state information (CSI) according to the signals; and
calculating an angle of arrival of the signals according to the CSI by the processor.

8. The method for signal sensing according to claim 7, wherein the step of synchronizing the receivers by receiving the time synchronization signal from the time server comprises:
configuring the receivers to a client mode when booted up and receiving the time synchronization signal from the time server by a network time protocol.

9. The method for signal sensing according to claim 8, wherein the step of monitoring the plurality of signals of the object comprises:
configuring the receivers to a sniffer mode from the client mode to monitor the signals, wherein the signals comprises a plurality of CSI packets having time information.

10. The method for signal sensing according to claim 9, wherein the step of calculating the angle of arrival of the signals comprises:
calculating the angle of arrival according to the CSI packets received by the receivers within a predetermined time interval.

11. The method for signal sensing according to claim 7, wherein the step of calculating the angle of arrival of the signals comprises:
calculating the angle of arrival by an algorithm, the algorithm comprising a MUSIC algorithm and a phase difference algorithm.

12. The method for signal sensing according to claim 7, wherein each of the receivers is an Orthogonal Frequency Division Multiplexing (OFDM) receiver.
